(19) United States
(12) Patent Application Publication
(10) Pub. No.: US 2026/0335052 A1
(43) Pub. Date: Oct. 29, 2026

(54) DENTAL HEALING ABUTMENT

(71) Applicant: INNODEN CO., LTD., Siheung-si, Gyeonggi-do (KR)

(72) Inventor: Cheon Seok JANG, Ansan-si, Gyeonggi-do (KR)

(21) Appl. No.: 19/124,228

(22) PCT Filed: Nov. 7, 2023
(86) PCT No.: PCT/KR2023/017718

§ 371 Date: Apr. 24, 2025

(30) Foreign Application Priority Data
Nov. 8, 2022 (KR) 10-2022-0147983

(51) Int. Cl. A61C 8/00

(52) U.S. Cl. CPC A61C 8/008; A61C 8/0013; A61C 8/0068

(57) ABSTRACT

The present disclosure relates to a healing abutment which is temporarily fixed to a fixture implanted in an alveolar bone while gums heal, before an abutment to be covered by a crown, which is an artificial tooth, is fitted. More specifically, the present disclosure relates to a dental healing abutment having a surface coated with a non-stick membrane made of a fluorine resin material having non-stick properties, or having an upper portion covered by a non-stick cap, so as to prevent foreign substances, such as food, from easily sticking.

### TECHNICAL FIELD

The present disclosure relates to a healing abutment which is temporarily fixed to a fixture implanted in an alveolar bone while gums heal, before an abutment to be covered by a crown, which is an artificial tooth, is fitted. More specifically, the present disclosure relates to a dental healing abutment having a surface coated with a non-stick membrane made of a fluorine resin material having non-stick properties, or having an upper portion covered by a non-stick cap, so as to prevent foreign substances, such as food, from easily sticking.

### BACKGROUND ART

A dental implant structure basically includes a fixture that is implanted into an alveolar bone, an abutment (support pole) that is fixed to the fixture and supports lateral or horizontal pressure according to occlusal force applied during mastication, and a crown (artificial tooth) that covers and restores the upper portion of the abutment to provide aesthetic beauty similar to that of a natural tooth.

The surgical procedure for dental implants with this structure is briefly explained below.

The fixture is implanted into the alveolar bone of a patient. Afterwards, the gums are cut so that the desired shape of the abutment can be fit according to an oral environment of the patient, and a healing abutment that forms the gums to the desired shape of the abutment is fitted. After about 2 to 4 weeks, or when the fixture is implanted and the healing abutment is immediately coupled and fixed, the healing abutment remains in the oral cavity for more than 2 months, during which time the gums have a recovery period. After the gum recovery period has passed, the healing abutment is separated from the fixture, the manufactured abutment is coupled, and a crown is placed on upper portion of the abutment to complete the procedure.

### PRIOR ART LITERATURE

### Patent Literature

(Patent Literature 1) KR 10-2082414 B1 (Feb. 21, 2020)

(Patent Literature 2) KR 10-1779111 B1 (Sep.11, 2017)

(Patent Literature 3) KR 10-2017-0106064 A (Sep. 20, 2017)

### DISCLOSURE

### Technical Problem

FIG. 1 is a view schematically illustrating a state in which a healing abutment according to a conventional technique is fastened and fixed to a fixture implanted in an alveolar bone. As illustrated in FIG. 1, a healing abutment 2 stabilizes gums 1 to heal the gums 1 and protects the space inside the gums 1 to prevent foreign substances or bacteria from entering the gums 1 or the alveolar bone.

As illustrated in FIG. 1, in the healing abutment 2, an upper portion is positioned at the upper portion of the gums 1 and exposed inside an oral cavity in a state of being coupled to a fixture 3 implanted in the alveolar bone. As a result, the healing abutment 2 is placed in an environment where it is easily exposed to food and contaminated when consuming food, or the like. In particular, the healing abutment has been manufactured using metal materials such as titanium in the past, but metal materials such as titanium have the characteristics of easily sticking food and easily accumulating dental plaque. This has caused inflammation around the healing abutment.

Accordingly, the present disclosure has been proposed to solve the various problems of related art, and an object (purpose) of the present disclosure is to provide a dental healing abutment having a surface coated with a non-stick membrane made of a fluorine resin material having non-stick properties, or having an upper portion covered by a non-stick cap, so as to prevent foreign substances, such as food, from easily sticking.

Furthermore, the present disclosure is not limited to the objects described above, and various other objects may be additionally provided through the techniques described in the embodiments and claims described below.

### Technical Solution

In order to achieve the above object, the present disclosure provides a dental healing abutment that is temporarily coupled and fixed to a fixture implanted in an alveolar bone while gums heal, in which a non-stick membrane made of fluorine resin material is formed on an outer surface of the dental healing abutment or a non-stick cap made of fluorine resin material covers the outer surface.

In addition, the dental healing abutment may include a coupling portion coupled and fixed to the fixture, and a body portion formed on the upper portion of the coupling portion.

Additionally, the non-stick membrane may be formed to cover the outer surface of the body portion.

Moreover, the non-stick cap may cover the outer surface of the body portion.

Additionally, in the body portion, a diameter of the upper portion may be larger than the diameter of the lower portion.

In addition, the coupling portion may be screw-coupled to the fixture.

Additionally, a driver groove may be formed in the upper portion of the body portion.

Furthermore, the non-stick cap may have a through hole formed in a portion corresponding to the driver groove.

Moreover, a peripheral groove may be formed around a periphery of the body portion.

In addition, a dental barrier film installed to cover a graft to prevent detachment of the graft transplanted to a defect area of the alveolar bone may be coupled to the peripheral groove.

In addition, the non-stick cap includes a locking jaw, which is inserted into a locking groove formed on the periphery of the body portion and then locked to the locking groove so that the non-stick cap is prevented from being separated from the body portion.

### Advantageous Effects

As described above, according to the dental healing abutment according to the present disclosure, the non-stick membrane made of the fluorine resin material having non-stick properties is formed on the surface, or the non-stick cap is installed to cover the surface, thereby preventing foreign substances such as food from easily sticking to the surface, or preventing dental plaque from easily accumulating in advance, thereby fundamentally preventing inflammation from occurring around the healing abutment.

In addition, according to the dental healing abutment according to the present disclosure, since the non-stick membrane made of the fluorine resin material having non-stick properties is formed on the surface, or the non-stick cap is installed to cover the surface, excellent heat resistance, chemical resistance, and weather resistance are provided, sterilization and disinfection are possible without changes in the properties or appearance of the abutment even when using a high-pressure sterilizer in a dental clinic.

### DESCRIPTION OF DRAWINGS

FIG. 1 is a view illustrating a state in which a healing abutment according to the prior art is fixed to a fixture.

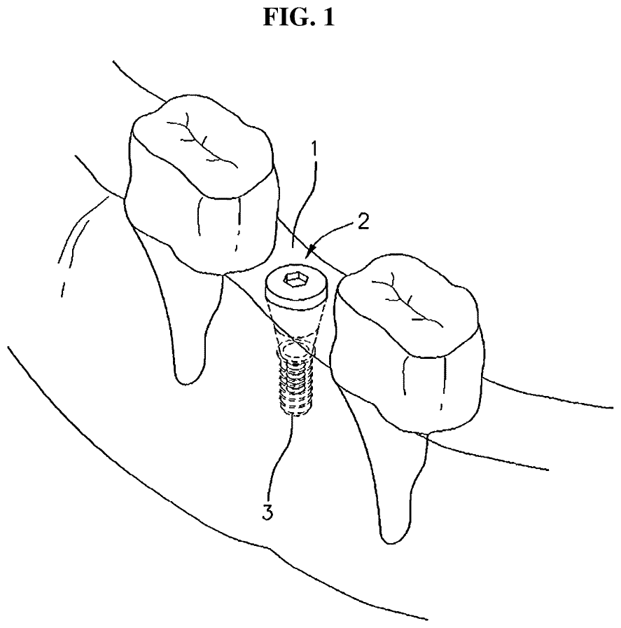

FIG. 2 is a view illustrating a dental healing abutment according to a first embodiment of the present disclosure.

FIG. 3 is a cross-sectional view of an installation state of the dental healing abutment illustrated in FIG. 2.

FIG. 4 is a view illustrating a dental healing abutment according to a second embodiment of the present disclosure.

FIG. 5 is a view illustrating a dental healing abutment according to a third embodiment of the present disclosure.

FIG. 6 is a view illustrating a dental healing abutment according to a fourth embodiment of the present disclosure.

FIG. 7 is a view illustrating a dental healing abutment according to a fifth embodiment of the present disclosure.

### BEST MODE

Hereinafter, the advantages and features of the present disclosure and the methods for achieving them will be clarified by referring to the embodiments described in detail below together with the attached drawings. In addition, the same reference numerals refer to the same components throughout the present specification. In addition, the respective components illustrated in each drawing may be excessively illustrated in size and shape, but this is for the convenience of explanation and is not intended to be limiting. In addition, when it is described as “A and/or B”, it may mean both A and B, or it may mean either A or B.

Hereinafter, embodiments of the present disclosure will be described in detail with reference to the attached drawings.

### First Embodiment

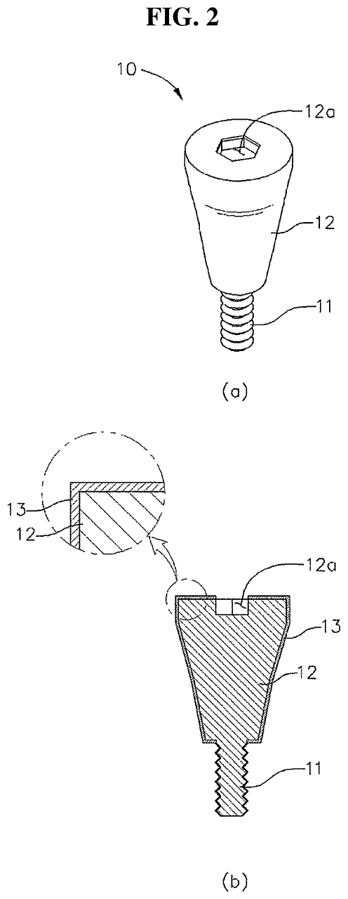

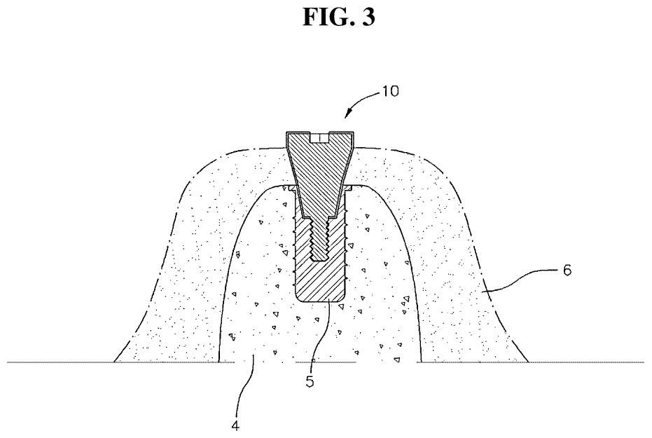

FIG. 2 is a view illustrating a dental healing abutment according to a first embodiment of the present disclosure, and FIG. 3 is a cross-sectional view of an installation state of the dental healing abutment illustrated in FIG. 2.

Referring to FIGS. 2 and 3, a dental healing abutment 10 according to the first embodiment of the present disclosure is a healing abutment that is temporarily coupled and fixed to a fixture 5 implanted in an alveolar bone 4 while gums 6 heal, and a non-stick membrane 13 made of fluorine resin material having non-stick properties is formed on the outer surface to prevent foreign substances such as food from easily sticking to the outer surface.

The non-stick membrane 13 may be formed by coating the outer surface of the dental healing abutment 10 with a fluorine resin material having non-stick properties to prevent foreign substances such as food from sticking thereto. The fluorine resin is a synthetic resin obtained by polymerizing an olefin containing fluorine, and has excellent heat resistance, chemical resistance, and weather resistance, and has excellent non-stick properties and lubricity compared to other materials. In addition, the fluorine resin enables sterilization (disinfection) without a change in properties even in a high-pressure sterilizer used in dentistry. For example, the fluorine resin may be a resin containing fluorine such as polytetrafluoroethylene (PTFE), fluorinated ethylene propylene copolymer (FEP), perfluoroalkoxy (PFA), polyvinylidene Fluoride (PVDF), poly ether ketone (PEEK), ethylene tetra fluoro ethylene (ETFE), or the like. In addition, all known fluorine-containing resins may be used.

The dental healing abutment 10 according to the first embodiment of the present disclosure includes a coupling portion 11 coupled to a fixture 5, and a body portion 12 formed above the coupling portion 11. The coupling portion 11 and the body portion 12 are base bodies of the dental healing abutment 10, and may be made of a metal material such as titanium to have a predetermined strength so as not to be easily broken, or may use engineering plastics that can replace metal.

The non-stick membrane 13 may be formed over the entire outer surface of the coupling portion 11 and the body portion 12, as illustrated in FIG. 2B, or may be formed only on the outer surface of the body portion 12 excluding the coupling portion 11. Alternatively, as illustrated in FIG. 3, the non-stick membrane 13 may be formed only on the exposed surface of the body portion 12 that is exposed to the upper side of the gums 6.

The coupling portion 11 may be screw-connected to the fixture 5 implanted in the alveolar bone 4, as illustrated in FIG. 3. For example, the coupling portion 11 may have a male screw portion formed on the outer surface so as to be screw-connected to a female screw portion formed in the center of the fixture 5. It is preferable that the coupling portion 11 be formed integrally with the body portion 12, as illustrated in FIG. 2, but this is only an example, and the coupling portion and the body portion may be manufactured independently of each other and then assembled using a screw-coupling method, or the like.

As illustrated in FIG. 3, a part of the upper portion of the body portion 12 is exposed (protruded) upward from the gums 6 in a state where the coupling portion 11 is coupled to the fixture 5. In addition, a driver groove 12a is formed on the upper surface of the body portion 12 exposed upward from the gums 6 into which a driver tool (not illustrated) is inserted to assemble or disassemble the dental healing abutment 10 to the fixture 5.

The driver groove 12a of the body portion 12 into which the driver tool is inserted may have a hexagonal, straight or cross-shaped structure. In addition, the drive groove may have various other shapes.

In addition, the body portion 12 may be formed so that the diameter of the lower portion is smaller than the diameter of the upper portion. For example, the body portion 12 has an increasing diameter from the lower portion, that is, the upper end of the coupling portion 11, to the upper portion.

### Second Embodiment

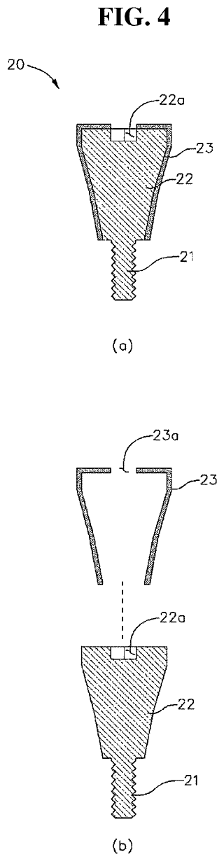

FIG. 4 is a view schematically illustrating a dental healing abutment according to a second embodiment of the present disclosure.

Referring to FIG. 4, a dental healing abutment 20 according to the second embodiment of the present disclosure includes a coupling portion 21 and a body portion 22, similar to the first embodiment. However, in the second embodiment, unlike the first embodiment, instead of the non-stick membrane being formed on the outer surface of the body portion 22 by coating, a non-stick cap 23 independently manufactured using a fluorine resin material is covered on the body portion 22 to add non-stick properties to the outer surface of the healing abutment 20.

The non-stick cap 23 is formed of a fluorine resin material, similar to the non-stick membrane 13 according to the first embodiment. As illustrated in FIG. 4B, the non-stick cap 23 has a through hole 23a formed in the upper portion of the body portion 22 so that the driver groove 22a formed in the upper portion is exposed to the outside. In this case, the non-stick cap 23 may have elasticity so as to be closely attached to the outer surface of the body portion 22, and for this purpose, a fluorine resin material having elasticity may be used.

Meanwhile, in FIG. 4, the non-stick cap 23 is installed to cover the outer surface of the body portion 22 excluding the coupling portion 21, but this is only as an example. The non-stick cap may be extended to the outer surface of the coupling portion 21.

### Third Embodiment

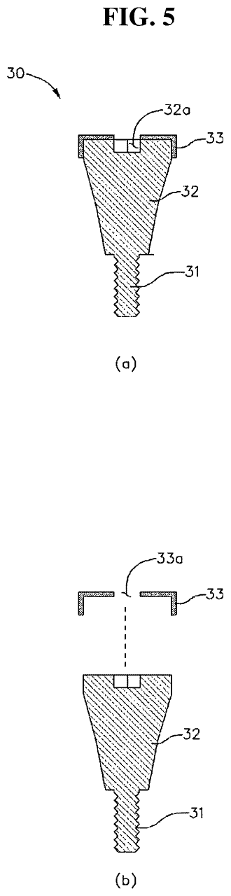

FIG. 5 is a view schematically illustrating a dental healing abutment according to a third embodiment of the present disclosure.

Referring to FIG. 5, a dental healing abutment 30 according to the third embodiment of the present disclosure includes a coupling portion 31, a body portion 32, and a non-stick cap 33, similar to the second embodiment. However, in the third embodiment, unlike the second embodiment, the non-stick cap 33 is formed to cover the exposed surface of the body portion 32 exposed upward from the gums, rather than covering the entire outer surface of the body portion 32.

The non-stick cap 33 is also formed of fluorine resin material in the same manner as the non-stick cap 23 according to the second embodiment. In addition, the non-stick cap 33 has a through hole 33a formed so that the driver groove 32a formed in the body portion 32 is exposed to the outside, as illustrated in FIG. 5B.

### Fourth Embodiment

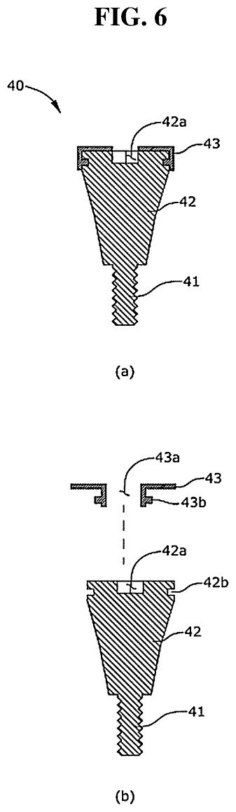

FIG. 6 is a view schematically illustrating a dental healing abutment according to a fourth embodiment of the present disclosure.

Referring to FIG. 6, a dental healing abutment 40 according to the fourth embodiment of the present disclosure includes a coupling portion 41, a body portion 42, and a non-stick cap 43, similar to the third embodiment. However, in the fourth embodiment, unlike the third embodiment, a locking jaw 43b is further formed as illustrated in FIG. 6B to prevent the non-stick cap 43 from being detached from the body portion 42, and correspondingly, a locking groove 42b into which the locking jaw 43b is inserted is formed around the outer surface of the body portion 42.

### Fifth Embodiment

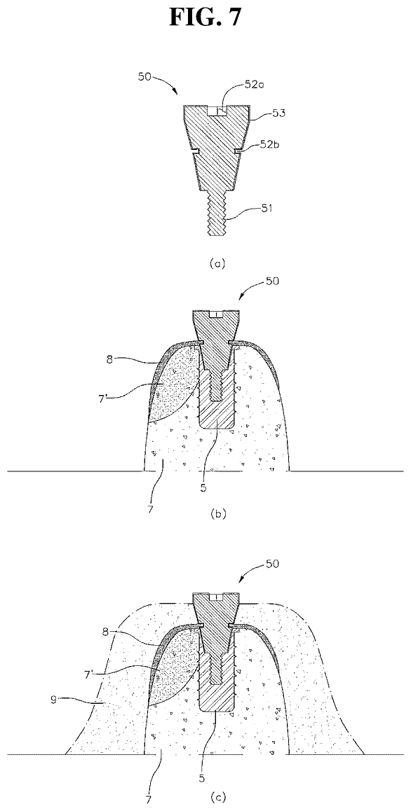

FIG. 7 is a view schematically illustrating a dental healing abutment according to a fifth embodiment of the present disclosure.

Referring to FIG. 7, a dental healing abutment 50 according to the fifth embodiment of the present disclosure includes a coupling portion 51, a body portion 52, and a non-stick membrane 53, similar to the first embodiment. However, in the fifth embodiment, unlike the first embodiment, a peripheral groove 52b into which a dental barrier film 8 is inserted is formed around the upper portion of the body portion 52, as illustrated in FIG. 7A and B and other than that, it may be formed with the same structure as the coupling portion 11 and the body portion 12 according to the first embodiment.

The dental barrier film 8 is formed to cover the transplanted area after filling the area where the alveolar bone 7 is missing with a graft (artificial bone, or the like) 7′, thereby preventing detachment of the transplanted graft 7′ and preventing gums 9 from growing and entering the defect area of the alveolar bone 7, thereby allowing the missing alveolar bone 7 to be completely restored, covering the alveolar bone 7 and the graft 7′. This dental barrier film 8 may be divided into two types: an absorbable barrier film and a non-absorbable barrier film.

The absorbable barrier film has the advantage of not needing to be removed because the adsorbable barrier film is absorbed over time after use. However, the adsorbable barrier film has the disadvantage of being difficult to obtain the desired alveolar bone when the shape of the adsorbable barrier film is not maintained during the period in which the alveolar bone is completely regenerated. The non-absorbable barrier film has the advantage of being able to obtain the desired alveolar bone shape because the non-absorbable barrier film does not dissolve and absorb over time, but has the disadvantage of having to be removed through additional removal surgery. Despite these disadvantages, the non-absorbable barrier film is mainly used to obtain the desired alveolar bone shape.

Among these, the non-absorbable barrier film should satisfy the following conditions. First, the non-absorbable barrier film should not be absorbed. This is because the transplanted graft should maintain the original shape and the alveolar bone should be restored in response to that shape. Second, the non-adsorbable barrier film should not dissolve and be absorbed over time. Third, the non-absorbable barrier film should not induce an immune response in the human body. To meet these conditions, the conventional non-absorbable barrier film uses a relatively hard (rigid) material.

In the fifth embodiment of the present disclosure, the non-absorbable barrier film may be used as the dental barrier film 8, and the non-absorbable barrier film may be titanium mesh, fluorine resin material, silicone rubber, or synthetic rubber. In this case, the synthetic rubber may be fluorine rubber containing fluorine. For example, the fluorine rubber may be fluoro elastomers (FKM) or perfluoro Elastomers (FFKM). In addition, the dental barrier film 8 may be formed as a single-layer film or a laminated film of two or more layers. In addition, a single metal frame or an alloy metal frame having bendability may be installed on the dental barrier film 8.

As illustrated in FIG. 7B, after filling the defect area of the alveolar bone 7 with the graft 7′, the dental barrier film 8 covers the alveolar bone 7 and the graft 7′. The dental barrier film 8 has a through hole formed through which the dental healing abutment 50 according to the fifth embodiment of the present disclosure passes. The through hole may be larger than the diameter of the body portion 52 and smaller than the diameter of the peripheral groove 52b. Thereafter, the coupling portion 51 of the dental healing abutment 50 according to the fifth embodiment of the present disclosure is inserted into a female screw portion formed in the center of the fixture 5 through the through hole formed in the dental barrier film 8, and then rotated in the tightening direction. Accordingly, the dental healing abutment is stably fastened and fixed to the fixture 5.

While the preferred embodiments of the present disclosure have been described and illustrated using specific terms, such terms are only intended to clearly describe the present disclosure. It should be understood that the embodiments and terms described herein may be modified and altered in various ways without departing from the spirit and scope of the following claims. Such modified embodiments should not be construed individually from the spirit and scope of the present disclosure, but should fall within the scope of the claims of the present disclosure.

### REFERENCE SIGN LIST

1, 6, 9: gums
2, 10, 20, 30, 40, 50: healing abutment
3, 5: fixture
4, 7: alveolar bone
7′: graft
8: dental barrier film
11, 21, 31, 41, 51: coupling portion
12, 22, 32, 42, 52: body portion
12a, 22a, 32a, 42a, 52a: driver groove
13, 53: non-stick membrane
23, 33, 43: non-stick cap
23a, 33a, 43a: through hole
42b: locking groove
43b: locking jaw
52b: peripheral groove

1. A dental healing abutment that is temporarily coupled and fixed to a fixture implanted in an alveolar bone while gums heal,
wherein a non-stick membrane made of a fluorine resin material is formed on an outer surface of the dental healing abutment or the non-stick cap made of the fluorine resin material covers the outer surface.

2. The dental healing abutment of claim 1 comprising:
a coupling portion coupled and fixed to the fixture; and
a body portion formed on the upper portion of the coupling portion.

3. The dental healing abutment of claim 2, wherein the non-stick membrane is formed to cover an outer surface of the body portion.

4. The dental healing abutment of claim 2, wherein the non-stick cap covers an outer surface of the body portion.

5. The dental healing abutment of claim 2, wherein in the body portion, a diameter of the upper portion is larger than a diameter of the lower portion.

6. The dental healing abutment of claim 2, wherein the coupling portion is screw-coupled to the fixture.

7. The dental healing abutment of claim 2, wherein a driver groove is formed in the upper portion of the body portion.

8. The dental healing abutment of claim 7, wherein the non-stick cap has a through hole formed in a portion corresponding to the driver groove.

9. The dental healing abutment of claim 2, wherein a peripheral groove is formed around a periphery of the body portion.

10. The dental healing abutment of claim 9, wherein a dental barrier film installed to cover a graft to prevent detachment of the graft transplanted to a defect area of the alveolar bone is coupled to the peripheral groove.

11. The dental healing abutment of claim 2, wherein the non-stick cap includes a locking jaw, which is inserted into a locking groove formed on the periphery of the body portion and then locked to the locking groove so that the non-stick cap is prevented from being separated from the body portion.
